(19) United States
(12) Patent Application Publication
(10) Pub. No.: US 2024/0385094 A1
(43) Pub. Date: Nov. 21, 2024

(54) METHOD FOR PROVIDING INFORMATION, ANALYSIS SYSTEM AND PROGRAM

(71) Applicant: SHIMADZU CORPORATION, Kyoto (JP)

(72) Inventor: Tomohisa HASUNUMA, Kobe-shi, Hyogo (JP)

(73) Assignee: SHIMADZU CORPORATION, Kyoto (JP)

(21) Appl. No.: 18/686,188

(22) PCT Filed: Mar. 4, 2022
(86) PCT No.: PCT/JP2022/009353

§ 371 Date: May 17, 2024

(30) Foreign Application Priority Data
Aug. 31, 2021 (JP) 2021-141440

(51) Int. Cl. G01N 5/00; C12Q 1/02

(52) U.S. Cl. CPC G01N 5/00; C12Q 1/02

(57) ABSTRACT

An information processing apparatus obtains a weight of a metabolite specified about a culture solution in a vessel, and obtains a weight of a microorganism specified about the culture solution in the vessel. The information processing apparatus derives an index about the weight of the metabolite using the weight of the metabolite and an inverse of the weight of the microorganism, and outputs the index.

### TECHNICAL FIELD

The present disclosure relates to a method for providing information about a metabolite, an analysis system and a program.

### BACKGROUND ART

As disclosed in PTL 1, there is known an apparatus that cultures cells of microorganisms or the like by adjusting the dissolved oxygen concentration while stirring a culture solution in a vessel.

### CITATION LIST

### Patent Literature

PTL 1: International Publication No. 2020/017407

### SUMMARY OF INVENTION

### Technical Problem

A weight of a metabolite of a microorganism may be used for analysis of the microorganism. In such a case, there has been a demand for such a technique that a researcher handles a weight of a metabolite in consideration of an environment where the metabolite is produced.

The present disclosure has been made in order to solve the above-described problem, and an object thereof is to provide a technique for handling a weight of a metabolite in consideration of an environment where the metabolite is produced.

### Solution to Problem

A first aspect of the present disclosure relates to a method for providing information about a metabolite, the method including: obtaining a weight of the metabolite specified about a culture solution in a vessel; obtaining a weight of a microorganism specified about the culture solution in the vessel, deriving an index about the weight of the metabolite using the weight of the metabolite and an inverse of the weight of the microorganism; and outputting the index.

A second aspect of the present disclosure relates to an analysis system that provides information about a weight of a metabolite of a microorganism, the analysis system including an information processing apparatus, wherein the information processing apparatus obtains a weight of the metabolite specified about a culture solution of the microorganism in a vessel, obtains a weight of the microorganism specified about the culture solution, derives an index about the weight of the metabolite using the weight of the metabolite and an inverse of the weight of the microorganism, and outputs the index.

A third aspect of the present disclosure relates to a program executed by a computer that provides information about a metabolite, the program causing the computer to perform: obtaining a weight of the metabolite specified about a culture solution in a vessel; obtaining a weight of a microorganism specified about the culture solution in the vessel; deriving an index about the weight of the metabolite using the weight of the metabolite and an inverse of the weight of the microorganism; and outputting the index.

### Advantageous Effects of Invention

According to the present disclosure, information for handling a weight of a metabolite in consideration of an environment where the metabolite is produced is provided.

### BRIEF DESCRIPTION OF DRAWINGS

FIG. 1 is a block diagram showing a schematic configuration of an automated pretreatment system.

FIG. 2 is a channel diagram showing a channel configuration of a sampling apparatus.

FIG. 3 is a block diagram showing a schematic configuration of a control device.

FIG. 4 shows schemes for obtaining a weight of a metabolite and a weight of a microorganism about a culture solution in a cell culturing apparatus 100.

FIG. 5 is a flowchart of a process for outputting an index about the weight of the metabolite

FIG. 6 shows a volume of a liquid supplied to a tube to measure the weight of the microorganism and a volume of a liquid supplied to a tube to measure the weight of the metabolite in an analysis system 700.

### DESCRIPTION OF EMBODIMENTS

The present embodiment will be described in detail with reference to the drawings, in which the same or corresponding portions are denoted by the same reference characters and description thereof will not be repeated in principle.

### Schematic Configuration of Analysis System

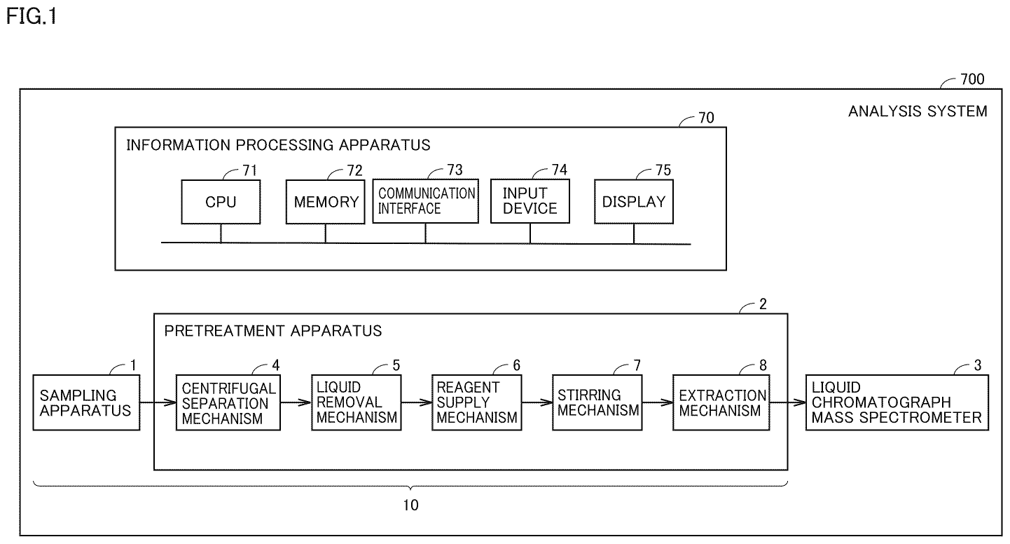

FIG. 1 is a block diagram showing a schematic configuration of an analysis system 700. Analysis system 700 includes a liquid chromatograph mass spectrometer 3, an automated pretreatment system 10 and an information processing apparatus 70. Automated pretreatment system 10 is an apparatus for automatically performing pretreatment on an analyte of liquid chromatograph mass spectrometer 3. One example of the analyte is a cultured microorganism. Another example is a metabolite of a microorganism. Information processing apparatus 70 can communicate with automated pretreatment system 10 and liquid chromatograph mass spectrometer 3, and comprehensively controls analysis system 700.

Information processing apparatus 70 includes a central processing unit (CPU) 71, a memory 72, a communication interface 73, an input device 74, and a display 75. Memory 72 includes, for example, a read only memory (ROM) and a random access memory (RAM), and can non-temporarily store various types of data including a control program.

Communication interface 73 is implemented by, for example, a network card and causes information processing apparatus 70 to communicate with automated pretreatment system 10 and liquid chromatograph mass spectrometer 3. Input device 74 inputs data input by a user to CPU 71 and is implemented by, for example, a keyboard and/or a mouse. Display 75 displays a result of computation by CPU 71 and is implemented by, for example, a liquid crystal display.

Automated pretreatment system 10 includes a sampling apparatus 1 and a pretreatment apparatus 2. In automated pretreatment system 10, a metabolite of a microorganism on which pretreatment has been performed is extracted from the microorganism. The extracted metabolite is supplied to liquid chromatograph mass spectrometer 3. Liquid chromatograph mass spectrometer 3 is merely an example of an analysis apparatus for analyzing an analyte. The analyte can also be analyzed using another analysis apparatus.

Sampling apparatus 1 is an apparatus for sampling a liquid from a vessel (culture vessel). For example, a microorganism is cultured in a culture solution containing a culture medium in a vessel called “bioreactor”. In the bioreactor, a stirring member rotated using the magnetic force, an oxygen concentration sensor for detecting the concentration of dissolved oxygen, and the like are provided, for example By adjusting the dissolved oxygen concentration while stirring the culture solution containing the culture medium and the microorganism in the bioreactor, the microorganism is cultured in sampling apparatus 1.

Pretreatment apparatus 2 performs pretreatment on the microorganism included in the culture solution (culture sample) sampled from the inside of the bioreactor. In sampling apparatus 1, the culture solution is housed in a tube. Pretreatment apparatus 2 includes a centrifugal separation mechanism 4, a liquid removal mechanism 5, a reagent supply mechanism 6, a stirring mechanism 7, and an extraction mechanism 8. These mechanisms sequentially perform pretreatment on the culture solution in the tube.

Centrifugal separation mechanism 4 provides the centrifugal force to the culture solution in the tube. As a result, the culture solution in the tube is separated into a solid component that sinks at the bottom of the tube and a liquid component that floats above the solid component, with a solid-liquid interface being a boundary. The solid component is a culture (e.g., cultured microorganism). The liquid component that floats above the solid component is a supernatant separated from the culture solution.

Liquid removal mechanism 5 suctions the supernatant from the tube. As a result, the liquid in the tube is removed and the microorganism remains in the tube.

Reagent supply mechanism 6 supplies a reagent for extracting the metabolite of the microorganism into the tube. As a result, a mixed solution of the microorganism and the reagent is generated in the tube.

Stirring mechanism 7 stirs the mixed solution. By stirring the mixed solution, a suspension including the metabolite of the microorganism is obtained.

Extraction mechanism 8 extracts a part of the suspension as an extract. The extract is supplied to liquid chromatograph mass spectrometer 3.

### <Schematic Configuration of Sampling Apparatus>

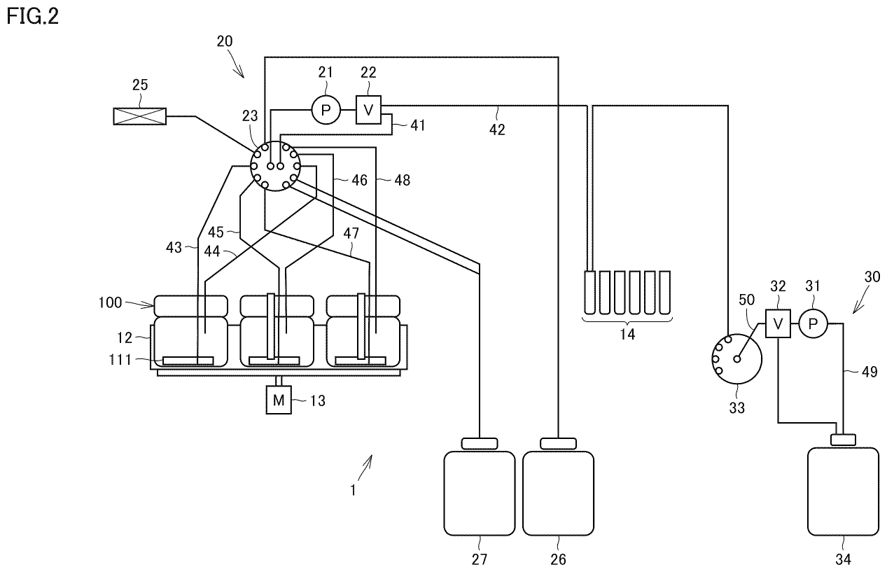

FIG. 2 is a channel diagram showing a channel configuration of sampling apparatus 1. In sampling apparatus 1, a culture solution including a microorganism in a cell culturing apparatus 100 called “bioreactor” is sampled. Cell culturing apparatus 100 includes therein a stirring element 111 as the stirring member rotated using the magnetic force.

Cell culturing apparatus 100 is held by a holding unit 12 provided in sampling apparatus 1. In the present embodiment, three cell culturing apparatuses 100 can be held by one holding unit 12, and a plurality of (e.g., four) such holding units 12 are provided. Only one holding unit 12 may be provided. Holding unit 12 may be configured to hold two or less or four or more cell culturing apparatuses 100.

Cell culturing apparatus 100 can perform culturing in a state of being heated by a heater (not shown) provided in holding unit 12. A motor 13 for rotating a magnet (not shown) is coupled to holding unit 12. By rotating motor 13, the magnet is rotated, and stirring element 111 in each cell culturing apparatus 100 can be rotated by the magnetic force.

In sampling apparatus 1, culturing can be performed by stirring the culture solution by stirring element 111, while controlling the temperature of the culture solution in cell culturing apparatus 100. In sampling apparatus 1, the culture solution including the cultured microorganism is sampled in a tube 14 at an arbitrary timing.

Sampling apparatus 1 includes a culture solution sampling mechanism 20 for sampling a culture solution in tube 14, and a reagent sampling mechanism 30 for sampling a reagent in tube 14. An example of the reagent is a reagent (quencher) for stopping the metabolic reaction of a microorganism in a culture solution. A mixed solution obtained by mixing the culture solution and the reagent is housed in tube 14, sealed by a cap (not shown), and conveyed to pretreatment apparatus 2. Tube 14 that houses only the culture solution (without being mixed with the reagent) may be conveyed to pretreatment apparatus 2.

Culture solution sampling mechanism 20 includes a pump 21 and a plurality of valves 22 and 23. Valve 23 has, for example, a pair of common ports and five pairs of (ten in total) selection ports, and the channel can be switched by arbitrarily selecting any one pair of selection ports and connecting the selected one pair of selection ports to the pair of common ports.

Pump 21 and valve 22 are provided in a channel 41 that connects the pair of common ports. Valve 22 constitutes a channel switching unit (first channel switching unit) for switching whether to guide the liquid in channel 41 to a branch channel 42 that branches off from channel 41. In other words, valve 22 can switch between a state in which the liquid flows between the pair of common ports through channel 41 and a state in which the liquid in channel 41 is guided to branch channel 42.

One pair of selection ports, of the five pairs of selection ports, are connected to an extraction channel 43 and an introduction channel 44 that communicate with one cell culturing apparatus 100, respectively. Extraction channel 43 is a channel for leading out the culture solution in cell culturing apparatus 100. On the other hand, introduction channel 44 is a channel through which the culture solution led out from cell culturing apparatus 100 through extraction channel 43 and circulating through channel 41 is again introduced into cell culturing apparatus 100. Another one pair of selection ports are connected to an extraction channel 45 and an introduction channel 46 that communicate with another cell culturing apparatus 100, respectively. Still another one pair of selection ports are connected to an extraction channel 47 and an introduction channel 48 that communicate with still another cell culturing apparatus 100, respectively.

In sampling apparatus 1, any one of extraction channels 43. 45 and 47 and a corresponding one of introduction channels 44, 46 and 48 are communicated with each other through channel 41 and pump 21 is driven in this state, and thereby, the culture solution in each cell culturing apparatus 100 can be circulated. In other words, channel 41, each of extraction channels 43, 45 and 47, and each of introduction channels 44, 46 and 48 constitute a circulation channel (first circulation channel) for circulating the culture solution in each cell culturing apparatus 100.

Pump 21 constitutes a circulation mechanism (first circulation mechanism) that circulates the culture solution in each cell culturing apparatus 100 through the first circulation channel by leading out the culture solution from each cell culturing apparatus 100 into the first circulation channel and introducing the culture solution from the first circulation channel into each cell culturing apparatus 100.

A leading end of each of extraction channels 43, 45 and 47 is immersed in the culture solution in corresponding cell culturing apparatus 100. On the other hand, a leading end of each of introduction channels 44, 46 and 48 is located at a position spaced upward from the culture solution in corresponding cell culturing apparatus 100. The culture solution led out from cell culturing apparatus 100 through extraction channels 43, 45 and 47 and circulating through channel 41 falls from the leading ends of introduction channels 44, 46 and 48 and is introduced into cell culturing apparatus 100.

In sampling apparatus 1, at least a portion where pump 21 is provided, of channel 41 that connects the pair of common ports, is formed of a flexible tube. Pump 21 is, for example, a tubing pump, and can deliver the liquid in the tube by deforming (compressing and relaxing) the flexible tube.

By switching valve 22 as the first channel switching unit provided in the middle of channel 41, the culture solution circulating into each cell culturing apparatus 100 through channel 41 can flow out to branch channel 42. At this time, a leading end of branch channel 42 is arranged in tube 14 and the culture solution is sampled in tube 14 through branch channel 42

One pair of selection ports, of the two pair of selection ports other than the three pair of selection ports to which extraction channels 43, 45 and 47 and introduction channels 44, 46 and 48 are connected, are connected to a cleaning liquid tank 26 and a waste liquid tank 27, respectively. The remaining one pair of selection ports are connected to a filer 25 and waste liquid tank 27, respectively. A cleaning liquid for cleaning the channel of the culture solution is housed in cleaning liquid tank 26.

When the culture solution is sampled from any one of cell culturing apparatuses 100 into tube 14 and then valve 23 is switched to connect cleaning liquid tank 26 and waste liquid tank 27 to channel 41, and pump 21 is driven in this state, the cleaning liquid in cleaning liquid tank 26 is discharged to waste liquid tank 27 through channel 41. As a result, channel 41, valve 22 and the like can be cleaned with the cleaning liquid.

When cleaning with the cleaning liquid is performed and then valve 23 is switched to connect filer 25 and waste liquid tank 27 to channel 41, and pump 21 is driven in this state, the air is introduced into channel 41 through filer 25 and is discharged to waste liquid tank 27 together with the water remaining in channel 41. As a result, the water can be removed from channel 41, valve 22 and the like.

Reagent sampling mechanism 30 includes a pump 31 and a plurality of valves 32 and 33. Valve 33 has, for example, one common port and a plurality of selection ports, and the channel can be switched by arbitrarily selecting any one of the selection ports and connecting the selected selection port to the common port.

Pump 31 and valve 32 are provided in a channel 49, both ends of which communicate with a reagent tank 34. A reagent to be mixed with the culture solution sampled in tube 14 is housed in reagent tank 34. Channel 49 constitutes a circulation channel (second circulation channel) for circulating the reagent in reagent tank 34. Pump 31 constitutes a circulation mechanism (second circulation mechanism) for circulating the reagent in reagent tank 34 through the second circulation channel by leading out the reagent from reagent tank 34 into the second circulation channel and introducing the reagent from the second circulation channel into reagent tank 34.

In sampling apparatus 1, at least a portion where pump 31 is provided, of channel 49 whose both ends are connected to reagent tank 34, is formed of a flexible tube. Pump 31 is, for example, a tubing pump, and can deliver the reagent in the tube by deforming (compressing and relaxing) the flexible tube.

Valve 32 constitutes a channel switching unit (second channel switching unit) for switching whether to guide the liquid in channel 49 to a branch channel 50 that branches off from channel 49. In other words, valve 32 can switch between a state in which the reagent in reagent tank 34 is circulated through channel 49 and a state in which the reagent in channel 49 is guided to branch channel 50.

By switching valve 32 as the second channel switching unit provided in the middle of channel 49 as described above, the reagent circulating into reagent tank 34 through channel 49 can flow out to branch channel 50. Branch channel 50 is connected to the common port of valve 33 and any one of the selection ports of valve 33 is connected to tube 14. Therefore, by causing the selection port connected to tube 14 to communicate with the common port, the reagent flowing out from channel 49 to branch channel 50 can be sampled in tube 14.

### <Schematic Configuration of Control Device>

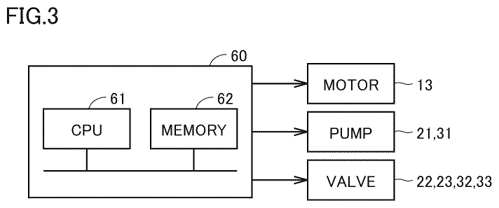

FIG. 3 is a block diagram showing a schematic configuration of a control device 60. Sampling apparatus 1 includes control device 60. Control device 60 includes, for example, a CPU 61 and a memory 62. Memory 62 includes, for example, a ROM and a RAM, and can non-temporarily store various types of data including a control program. CPU 61 can control the operation of motor 13, pumps 21 and 31, and valves 22, 23, 32, and 33, and the like by executing the control program stored in memory 62.

Control device 60 can circulate the culture solution in any one of cell culturing apparatuses 100 by driving pump 21 at a constant liquid delivery speed in a state where any one of extraction channels 43, 45 and 47 communicates with a corresponding one of introduction channels 44, 46 and 48 through channel 41. Control device 60 can sample the culture solution in channel 41 to tube 14 by switching valve 22 for a prescribed time period based on the control program to communicate channel 41 with branch channel 42.

Control device 60 can control an amount of sampling of the culture solution by controlling the time period during which the channel is switched by valve 22. In other words, when the liquid delivery speed of pump 21 is known in advance, a desired amount of the culture solution can be accurately sampled to tube 14 by adjusting the time period during which channel 41 and branch channel 42 are communicated with each other.

Control device 60 can circulate the reagent in reagent tank 34 by driving pump 31 at a constant liquid delivery speed in a state where one end of channel 49 communicates with the other end of channel 49. Control device 60 can sample the reagent in channel 49 to tube 14 by switching valve 32 for a prescribed time period based on the control program to communicate channel 49 with branch channel 50 and switching valve 33 to communicate branch channel 50 with tube 14.

Control device 60 can control an amount of sampling of the reagent by controlling the time period during which the channel is switched by valve 32. In other words, when the liquid delivery speed of pump 31 is known in advance, a desired amount of the reagent can be accurately sampled to tube 14 by adjusting the time period during which channel 49 and branch channel 50 are communicated with each other.

### <Obtainment of Weight of Metabolite and Weight of Microorganism>

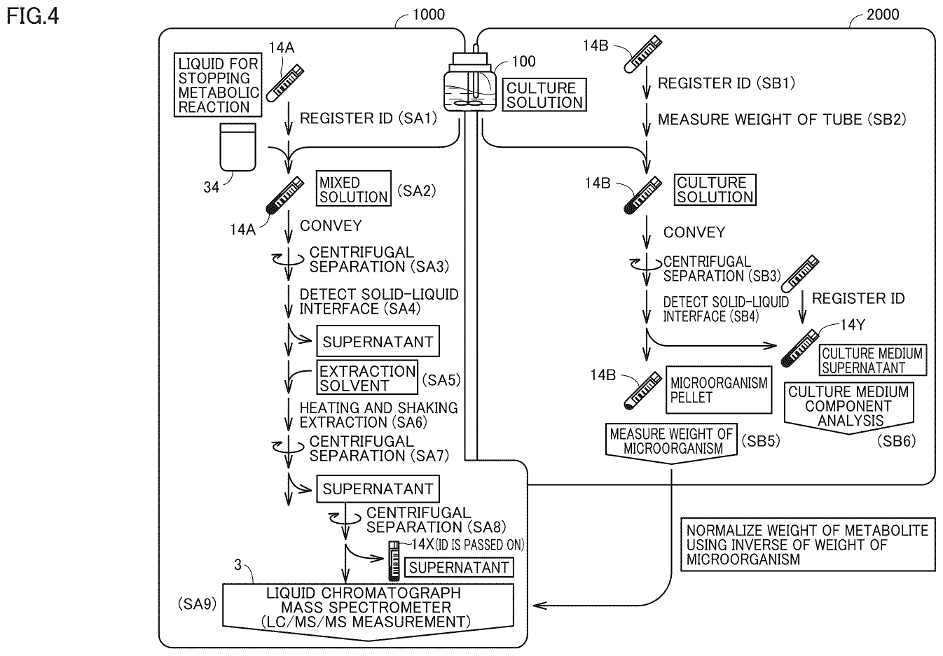

FIG. 4 is a diagram showing schemes for obtaining a weight of a metabolite and a weight of a microorganism about the culture solution in cell culturing apparatus 100. FIG. 4 shows a scheme 1000 for obtaining the weight of the metabolite and a scheme 2000 for obtaining the weight of the microorganism. Each of scheme 1000 and scheme 2000 will be described below.

### (Scheme 1000: Obtainment of Weight of Metabolite)

In scheme 1000, in step SA1, an ID given to a tube 14A is registered. In one implementation, the registration is implemented in information processing apparatus 70. More specifically, in information processing apparatus 70, when the ID given to tube 14A is input to input device 74 by the user, information that associates the ID with the tube to be processed (tube 14A) is registered in memory 72.

In step SA2, a mixed solution of the culture solution and a quencher is generated in tube 14A. More specifically, in sampling apparatus 1, the culture solution in cell culturing apparatus 100 and the quencher (liquid for stopping the metabolic reaction) in reagent tank 34 are supplied to tube 14A. Step SA2 is an example of quenching processing for stopping the metabolic reaction of the microorganism, which is performed on the culture solution. Thereafter, tube 14A is conveyed to pretreatment apparatus 2.

In step SA3, the centrifugal force is provided to the culture solution (mixed solution with the quencher) in tube 14A by centrifugal separation mechanism 4 of pretreatment apparatus 2.

In step SA4, a solid-liquid interface in tube 14A is detected and a supernatant is removed by liquid removal mechanism 5.

In step SA5, a reagent for extracting the metabolite of the microorganism is supplied into tube 14 by reagent supply mechanism 6. Thereafter, the mixed solution in tube 14A is stirred by stirring mechanism 7.

In step SA6, heating and shaking processing is performed on the mixed solution in tube 14A and then a part of the mixed solution is extracted by extraction mechanism 8.

In step SA7, the extracted mixed solution is centrifugally separated to obtain a supernatant, and in step SA8, the supernatant is further centrifugally separated. After the centrifugal separation in step SA8, the supernatant in tube 14A is housed in a tube 14X and forwarded to liquid chromatograph mass spectrometer 3. In information processing apparatus 70, the ID associated with tube 14A is passed on to tube 14X.

In step SA9, mass spectrometry is performed on the solution in tube 14X by liquid chromatograph mass spectrometer 3, and thereby, quantitative analysis of the metabolite about the culture solution introduced into tube 14A is performed.

Information processing apparatus 70 specifies the weight of the metabolite in the culture solution in tube 14A, using a result of quantitative analysis of the metabolite about the culture solution introduced into tube 14A. In one implementation, the weight of the metabolite in the culture solution in tube 14A is specified based on a ratio of a peak area corresponding to the metabolite to a peak area corresponding to an internal reference substance (e.g., 2-isopropylmalic acid) in a mass chromatogram obtained about the culture solution in tube 14A (solution in tube 14X). Information processing apparatus 70 registers the weight of the metabolite in memory 72 in association with the ID of tube 14A.

### (Scheme 2000: Obtainment of Weight of Microorganism)

In scheme 2000, in step SB1, an ID given to a tube 14B is registered. In one implementation, the registration is implemented in information processing apparatus 70. More specifically, in information processing apparatus 70, when the ID given to tube 14B is input to input device 74 by the user, information that associates the ID with the tube to be processed (tube 14B) is registered in memory 72.

In memory 72, the ID of tube 14B is associated with the ID of tube 14A. More specifically, the culture solution is supplied to tube 14B from the same cell culturing apparatus 100 as cell culturing apparatus 100 that supplied the culture solution to tube 14A. In analysis system 700, the IDs of tube 14A and tube 14B that are supplied with the culture solution from the same cell culturing apparatus 100 are associated with each other.

In step SB2, a weight of tube 14B is measured and registered in memory 72

Thereafter, in sampling apparatus 1, the culture solution is supplied to tube 14B. The culture solution supplied to tube 14B is from the same cell culturing apparatus 100 as cell culturing apparatus 100 that supplied the culture solution to tube 14A in step SA1 of scheme 1000. In other words, the culture solution is supplied to tube 14A and tube 14B from the same cell culturing apparatus 100. Thereafter, tube 14B is conveyed to pretreatment apparatus 2.

In step SB3, the centrifugal force is provided to the culture solution in tube 14B by centrifugal separation mechanism 4 of pretreatment apparatus 2.

In step SB4, a solid-liquid interface in tube 14B is detected and a supernatant is transported to another tube (tube 14Y) by liquid removal mechanism 5. An ID different from that of tube 14B is registered for tube 14Y and the supernatant transported to tube 14Y is subjected to culture medium component analysis (step SB6).

In step SB5, a weight of a pellet remaining in tube 14B after the transportation of the supernatant in step SB4 is measured. In one implementation, the user measures the weight of pellet-containing tube 14B and inputs a result of measurement to information processing apparatus 70 through input device 74. Information processing apparatus 70 obtains the weight of the pellet by subtracting the weight of tube 14B registered in step SB2 from the input weight. Then, information processing apparatus 70 registers, as the weight of the microorganism, the weight of the pellet in memory 72 in association with the ID of tube 14B. In another implementation, information processing apparatus 70 has a mechanism that measures the weight of pellet-containing tube 14B (not shown). Using this mechanism, information processing apparatus 70 measures the weight of pellet-containing tube 14B, obtains the weight of the pellet by subtracting the weight of tube 14B registered in step SB2 from the measured weight. and registers, as the weight of the microorganism, the weight of the pellet in memory 72 in association with the ID of tube 14B.

<Index about Weight of Metabolite>

In analysis system 700, information processing apparatus 70 obtains the weight of the metabolite for each tube, and further, outputs an index about the weight of the metabolite. The index about the weight of the metabolite is an index for indicating an amount of metabolite production per unit weight of the microorganism.

In order to derive this index, information processing apparatus 70 supplies the same amount (volume) of culture solution to two tubes from one cell culturing apparatus 100, specifies the weight of the metabolite about the culture solution supplied to one tube, specifies the weight of the microorganism about the culture solution supplied to the other tube, and calculates a product of the weight of the metabolite and an inverse of the weight of the microorganism. Information processing apparatus 70 obtains a value of the calculated product as the above-described index.

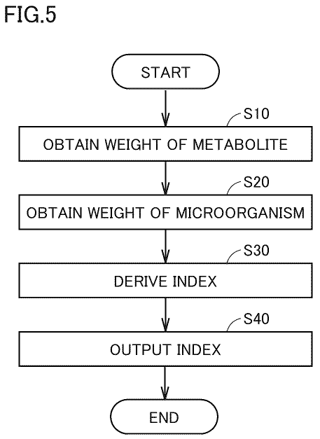

FIG. 5 is a flowchart of a process for outputting the index about the weight of the metabolite. In one implementation, in information processing apparatus 70, the process in FIG. 5 is performed by CPU 71 executing a given program. A flow of the process will be described below.

In step S10, information processing apparatus 70 reads, from memory 72, a weight of a metabolite associated with one tube (tube 14A). In one implementation, the weight of the metabolite may be obtained in accordance with scheme 1000 in FIG. 4 and registered in memory 72.

In step S20, information processing apparatus 70 reads, from memory 72, a weight of a microorganism associated with tube 14B. Tube 14B in step S20 is associated with tube 14A in step S10 in memory 72. In other words, the weight of the metabolite read in step S10 and the weight of the microorganism read in step S20 are about the culture solution provided from the same cell culturing apparatus 100.

In step S30, information processing apparatus 70 calculates an index as a product of the weight of the metabolite read in step S10 and an inverse of the weight of the microorganism read in step S20.

In step S40, information processing apparatus 70 causes display 75 to display the index calculated in step S30. Thereafter, information processing apparatus 70 ends the process in FIG. 5.

In the process in FIG. 5 described above, the index about the weight of the metabolite is derived as the product of the weight of the metabolite specified about the culture solution in the vessel (cell culturing apparatus 100) and the inverse of the weight of the microorganism specified about the culture solution. This index corresponds to a value derived by normalizing the weight of the metabolite with the weight of the microorganism. In addition, this index indicates an amount of metabolite production per unit weight of the microorganism. Therefore, it can be said that this index corresponds to information for handling the weight of the metabolite in consideration of an environment where the metabolite is produced (amount or number of the microorganism in the culture solution).

In the process described with reference to FIG. 5, the index is calculated and displayed. “Display” is an example of the way to output the index. Information processing apparatus 70 may output the calculated index by transmitting the calculated index to an external device.

### <Amount of Supply of Liquid for Measuring Weight of Microorganism and Amount of Supply of Liquid for Measuring Weight of Metabolite>

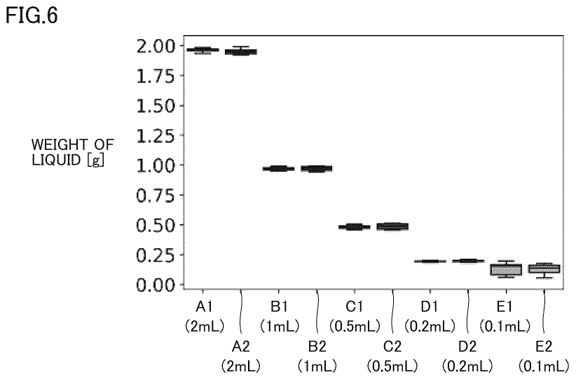

FIG. 6 shows a volume of a liquid supplied to a tube to measure the weight of the microorganism and a volume of a liquid supplied to a tube to measure the weight of the metabolite in analysis system 700. The vertical axis in FIG. 6 represents the volume of the liquid supplied to the tube.

The graph in FIG. 6 shows a result about each of ten groups (A1 to E2). For each group, the liquid is supplied to the tube six times. The graph in FIG. 6 shows a maximum value and a minimum value of the volume of the liquid supplied to the tube for each group. The results in FIG. 6 are results when pure water is used as an example of the liquid.

A target value (target volume) of the volume of the liquid supplied to the tube varies from group to group. The target volume for groups A1 and A2 is 2 mL, the target volume for groups B1 and B2 is 1 mL, the target volume for groups C1 and C2 is 0.5 mL, the target volume for groups D1 and D2 is 0.2 mL, and the target volume for groups E1 and E2 is 0.1 mL.

The numbers given to the respective groups indicate different purposes of supplying the liquid from cell culturing apparatus 100 to the tube in analysis system 700. “1” indicates that the liquid is supplied from cell culturing apparatus 100 to the tube (tube 14B) to measure the weight of the microorganism (scheme 2000 in FIG. 4). “2” indicates that the liquid is supplied from cell culturing apparatus 100 to the tube (tube 14B) to measure the weight of the metabolite (scheme 1000 in FIG. 4).

In the graph in FIG. 6, group A1 includes the maximum value of 2.00 g and the minimum value of 1.95 g. Group A2 includes the maximum value of 2.00 g and the minimum value of 1.93 g. In other words, group A1 and group A2 include the almost common maximum value and minimum value. Based on this, it can be said that the volume of the liquid supplied to tube 14B to measure the weight of the microorganism as group A1 is almost the same as the volume of the liquid supplied to tube 14A to measure the weight of the metabolite as group A2.

In addition, in the graph in FIG. 6, group B1 and group B2 include the almost common maximum value and minimum value. Furthermore, group C1 and group C2 include the almost common maximum value and minimum value, group D1 and group D2 include the almost common maximum value and minimum value, and group E1 and group E2 include the almost common maximum value and minimum value.

Therefore, it can be said that in analysis system 700, there is such a correlation that the volume of the liquid supplied to tube 14B to measure the weight of the microorganism is almost the same as the volume of the liquid supplied to tube 14A to measure the weight of the metabolite, when the target volume is the same. Therefore, it can be said that in analysis system 700, the weight of the metabolite is effectively normalized with (the inverse of) the weight of the microorganism measured using the culture solution supplied from the same cell culturing apparatus 100 as cell culturing apparatus 100 used to measure the weight of the metabolite.

In one implementation, in analysis system 700, in order to equalize the amount of the liquid supplied to the tube, control device 60 may stop stirring (or reduce the rotation speed for stirring) by stirring element 111 for a given time period immediately before the culture solution is supplied from cell culturing apparatus 100 to tube 14 (tube 14A, 14B). In another implementation, information processing apparatus 70 may instruct control device 60 to stop stirring (or reduce the rotation speed for stirring) and control device 60 may stop stirring (or reduce the rotation speed) in accordance with this instruction. An excessively long given time period may result in uneven culturing in the culture solution supplied to the tube, and an excessively short given time period may result in inhibition of equalization of the amount because bubbles for supplying oxygen to the culture solution supplied to the tube are included in the culture solution. In this sense, the given time period is approximately 2 to 10 minutes in one example, is preferably 3 to 7 minutes, and is more preferably 3 minutes.

### Aspects

It will be understood by those skilled in the art that the plurality of exemplary embodiments described above are specific examples of the following aspects.
(Clause 1) A method for providing information according to an aspect is a method for providing information about a metabolite. The method may include: obtaining a weight of the metabolite specified about a culture solution in a vessel; obtaining a weight of a microorganism specified about the culture solution in the vessel; deriving an index about the weight of the metabolite using the weight of the metabolite and an inverse of the weight of the microorganism; and outputting the index

According to the method for providing information described in clause 1, the information for handling the weight of the metabolite in consideration of an environment where the metabolite is produced is provided.
(Clause 2) In the method for providing information according to clause 1, the deriving the index may include deriving the index by calculating a product of the weight of the metabolite and the inverse of the weight of the microorganism.

According to the method for providing information described in clause 2, the index can be easily calculated.
(Clause 3) In the method for providing information according to clause 1 or 2, the weight of the metabolite may be specified about the culture solution on which quenching processing for stopping metabolic reaction of the microorganism has been performed.

According to the method for providing information described in clause 3, the progress of production of the metabolite after the culture solution is taken from the vessel to specify the weight of the metabolite can be avoided.
(Clause 4) An analysis system according to an aspect is an analysis system that provides information about a weight of a metabolite of a microorganism, the analysis system comprising an information processing apparatus, wherein the information processing apparatus may be configured to obtain a weight of the metabolite specified about a culture solution of the microorganism in a vessel, obtain a weight of the microorganism specified about the culture solution, derive an index about the weight of the metabolite using the weight of the metabolite and an inverse of the weight of the microorganism, and output the index.

According to the analysis system described in clause 4, the information for handling the weight of the metabolite in consideration of an environment where the metabolite is produced is provided.
(Clause 5) In the analysis system according to clause 4, the index may be derived by calculating a product of the weight of the metabolite and the inverse of the weight of the microorganism.

According to the analysis system described in clause 5, the index can be easily calculated.
(Clause 6) In the analysis system according to clause 4 or 5, the weight of the metabolite may be specified about the culture solution on which quenching processing for stopping metabolic reaction of the microorganism has been performed.

According to the analysis system described in clause 6, the progress of production of the metabolite after the culture solution is taken from the vessel to specify the weight of the metabolite can be avoided.
(Clause 7) A program according to an aspect is a program executed by a computer that provides information about a metabolite. The program may cause the computer to perform: obtaining a weight of the metabolite specified about a culture solution in a vessel; obtaining a weight of a microorganism specified about the culture solution in the vessel; deriving an index about the weight of the metabolite using the weight of the metabolite and an inverse of the weight of the microorganism; and outputting the index.

According to the program described in clause 7, the information for handling the weight of the metabolite in consideration of an environment where the metabolite is produced is provided.
(Clause 8) In the program according to clause 7, the deriving the index may include deriving the index by calculating a product of the weight of the metabolite and the inverse of the weight of the microorganism.

According to the program described in clause 8, the index can be easily calculated.
(Clause 9) In the program according to clause 7 or 8, the weight of the metabolite may be specified about the culture solution on which quenching processing for stopping metabolic reaction of the microorganism has been performed.

According to the program described in clause 9, the progress of production of the metabolite after the culture solution is taken from the vessel to specify the weight of the metabolite can be avoided

It should be understood that the embodiment disclosed herein is illustrative and non-restrictive in every respect. The scope of the present disclosure is defined by the terms of the claims, rather than the description of the embodiment above, and is intended to include any modifications within the scope and meaning equivalent to the terms of the claims.

### REFERENCE SIGNS LIST

1 sampling apparatus; 2 pretreatment apparatus; 3 liquid chromatograph mass spectrometer; 4 centrifugal separation mechanism; 5 liquid removal mechanism; 6 reagent supply mechanism; 7 stirring mechanism; 8 extraction mechanism; 10 pretreatment system; 12 holding unit; 13 motor; 14 tube; 20 culture solution sampling mechanism; 21, 31 pump; 22, 23, 32, 33 valve; 25 filter; 26 cleaning liquid tank; 27 waste liquid tank; 30 reagent sampling mechanism; 34 reagent tank; 41, 42, 49, 50 channel; 43, 45, 47 extraction channel; 44, 46, 48 introduction channel; 60 control device; 70 information processing apparatus; 100 cell culturing apparatus; 700 analysis system.

1. A method for providing information about a metabolite, the method comprising:
obtaining a weight of the metabolite specified about a culture solution in a vessel;
obtaining a weight of a microorganism specified about the culture solution in the vessel;
deriving an index about the weight of the metabolite using the weight of the metabolite and an inverse of the weight of the microorganism; and
outputting the index.

2. The method for providing information according to claim 1, wherein
the deriving the index includes deriving the index by calculating a product of the weight of the metabolite and the inverse of the weight of the microorganism.

3. The method for providing information according to claim 1, wherein
the weight of the metabolite is specified about the culture solution on which quenching processing for stopping metabolic reaction of the microorganism has been performed.

4. The method for providing information according to claim 3, wherein
the weight of the microorganism is specified about the culture solution on which the quenching processing has not been performed.

5. An analysis system that provides information about a weight of a metabolite of a microorganism, the analysis system comprising
an information processing apparatus, wherein
the information processing apparatus is configured to
obtain a weight of the metabolite specified about a culture solution of the microorganism in a vessel,
obtain a weight of the microorganism specified about the culture solution,
derive an index about the weight of the metabolite using the weight of the metabolite and an inverse of the weight of the microorganism, and
output the index.

6. The analysis system according to claim 5, wherein
the index is derived by calculating a product of the weight of the metabolite and the inverse of the weight of the microorganism.

7. The analysis system according to claim 5, wherein
the weight of the metabolite is specified about the culture solution on which quenching processing for stopping metabolic reaction of the microorganism has been performed.

8. The analysis system according to claim 7, wherein
the weight of the microorganism is specified about the culture solution on which the quenching processing has not been performed.

9. A non-transitory computer-readable storage medium storing a program executed by a computer that provides information about a metabolite, the program causing the computer to perform:
obtaining a weight of the metabolite specified about a culture solution in a vessel;
obtaining a weight of a microorganism specified about the culture solution in the vessel;
deriving an index about the weight of the metabolite using the weight of the metabolite and an inverse of the weight of the microorganism; and
outputting the index.

10. The non-transitory computer-readable storage medium according to claim 9, wherein
the deriving the index includes deriving the index by calculating a product of the weight of the metabolite and the inverse of the weight of the microorganism.

11. The non-transitory computer-readable storage medium according to claim 9, wherein
the weight of the metabolite is specified about the culture solution on which quenching processing for stopping metabolic reaction of the microorganism has been performed.

12. The non-transitory computer-readable storage medium according to claim 11, wherein
the weight of the microorganism is specified about the culture solution on which the quenching processing has not been performed.
